(19) United States
(12) Patent Application Publication
(10) Pub. No.: US 2016/0143866 A1
(43) Pub. Date: May 26, 2016

(54) Combination Therapy for Administration of Monoclonal Antibodies

(71) Applicant: Eli D. Ehrenpreis, Skokie, IL (US)

(72) Inventor: Eli D. Ehrenpreis, Skokie, IL (US)

(21) Appl. No.: 14/947,193

(22) Filed: Nov. 20, 2015

Related U.S. Application Data
(60) Provisional application No. 62/082,692, filed on Nov. 21, 2014.

(51) Int. Cl. A61K 31/165; A61K 9/00; A61K 39/395

(52) U.S. Cl. CPC A61K 31/165; A61K 39/3955; A61K 9/0019; A61K 9/0053; C07K 2317/21; C07K 2317/24; C07K 2317/55; A61K 2039/505

(57) ABSTRACT

A combination therapy is disclosed for the treatment of a patient, such as a human, with a monoclonal antibody. The methods can include administering to a patient a monoclonal antibody, for example, a TNT-alpha inhibitor such as adalimumab, certolizumab pegol, golimumab, and infliximab; and administering to the patient colchicine. The present teachings also provide a therapeutic combination and a kit including the therapeutic combination.

### CROSS REFERENCE TO RELATED APPLICATIONS

This application claims priority to and the benefit of the filing date of U.S. Provisional Patent Application No. 62/082,692, filed on Nov. 21, 2014, the entire disclosure of which is incorporated by reference herein.

### FIELD

The present teachings relate to the administration of monoclonal antibodies. More specifically, the present teachings relate to a therapeutic combination including a monoclonal antibody and colchicine and methods of administrating the same.

### BACKGROUND

The use of inhibitors of tumor necrosis factor (“TNF”) alpha has revolutionized the care of patients with Crohn's disease, ulcerative colitis, rheumatoid arthritis and other autoimmune disorders. Studies have shown that treatment with biologically-derived, monoclonal antibodies against TNF-alpha improves remission rates in patients with Crohn's disease, ulcerative colitis and rheumatoid arthritis when compared to immunomodulator therapy and other drugs. In addition, a clear benefit has been demonstrated with the use of monoclonal antibodies as maintenance therapy for inflammatory bowel disease (“IBD”) and other autoimmune conditions. Monoclonal antibodies are also used in the treatment of certain types of cancer and other disorders.

A significant problem that has developed with the use of monoclonal antibodies, particularly those that block TNF-alpha, is the formation of human anti-chimeric antibodies, or “HACAs.” HACAs appear to reduce the efficacy of monoclonal antibodies and predispose patients to develop acute and chronic infusion reactions. Lower serum trough concentrations of monoclonal antibodies, an indicator of diminished drug efficiency, have also been found to correlate with the presence of HACAs. Faster clearance of monoclonal antibodies from a patient's system, as reflected in measured levels (particularly the trough levels) of the monoclonal antibodies in the patient's blood, result in a diminution of their efficacy.

In an attempt to prevent the development of HACAs in a patient being treated with monoclonal antibodies, previous studies have examined combination therapy with drugs such as thiopurine agents, for example, azathiopurine or 6-mercaptopurine administered together with a monoclonal antibody. Although these studies have shown that combining monoclonal antibodies with thiopurines improve overall efficacy of the monoclonal antibody and reduce HACA formation, patients receiving a combination therapy of such agents are at increased risk for variety of opportunistic infections, lymphomas and other cancers, due to profound immunosuppression.

Therefore, there is a need to increase safely the time that a monoclonal antibody remains in the blood serum of a patient by decreasing its clearance from the patient's system, such as by reducing the formation of HACAs during treatment, without worsening the degree of immunosuppression caused by the monoclonal antibody. Such a therapy has the potential benefit of maximizing the effectiveness of the monoclonal antibody treatment and reducing the cost of drug administration.

### SUMMARY

In light of the foregoing, the present teachings provide a therapeutic combination and combination therapy for the treatment of autoimmune disorders, cancer, and other disorders treated with a monoclonal antibody. In particular, the methods include administration of a monoclonal antibody such as a TNF-alpha inhibitor and the anti-inflammatory agent, colchicine, where the therapeutic combination includes the monoclonal antibody and colchicine. The methods and compositions of the present teachings can address one or more of the deficiencies and/or disadvantages of the state-of-the art, for example, increased time that a monoclonal antibody remains in the blood serum, less frequent dosing, and little or no increase in immunosuppression.

Clinical studies have shown that colchicine is not effective as a treatment for IBD or rheumatoid arthritis. Thus, colchicine would not be expected to be effective as a treatment for autoimmune or other disorders in combination with a monoclonal antibody. However, without wishing to be bound to any particular theory, it is believed that colchicine unexpectedly increases the time that a monoclonal antibody remains in the blood serum of a patient by decreasing the clearance of the monoclonal antibody from the patient's system, for example, by reducing HACA formation. It is further believed that colchicine can disrupt microtubule polymerization or assembly. Colchicine also is believed to suppress nitric oxide synthetase 3 (eNOS3) genes in a patient undergoing monoclonal antibody therapy.

Accordingly, in one aspect, the present teachings provide methods of increasing the time that a monoclonal antibody such as a TNF-alpha inhibitor remains in the blood serum of a patient. Such methods can include administering to a patient a monoclonal antibody such as a TNF-alpha inhibitor, for example, adalimumab, certolizumab pegol, golimumab, and infliximab, and administering colchicine to the patient.

In various embodiments, the present teachings can provide methods of increasing the clearance time of a monoclonal antibody from the blood serum of a patient by disrupting microtubule polymerization or assembly in a patient undergoing monoclonal antibody therapy, whereby a monoclonal antibody concentration in the blood serum of the patient is lengthened compared to not disrupting microtubule polymerization or assembly.

In some embodiments, the present teachings provide methods of increasing the clearance time of a monoclonal antibody from the blood serum of a patient by suppressing nitric oxide synthetase 3 (eNOS3) genes in a patient including monoclonal antibody therapy, whereby a monoclonal antibody concentration in the blood serum of the patient is lengthened compared to not suppressing nitric oxide synthetase 3 (eNOS3) genes.

In certain embodiments, the present teachings can provide methods of increasing the clearance time of a monoclonal antibody from the blood serum of a patient by decreasing the formation or enhancing the removal of human anti-chimeric antibodies in a patient undergoing monoclonal antibody therapy, whereby a monoclonal antibody concentration in the blood serum of the patient is lengthened compared to not decreasing the formation or enhancing the removal of human anti-chimeric antibodies.

In another aspect, the present teachings provide a therapeutic combination including a monoclonal antibody such as a INF-alpha inhibitor and colchicine. The monoclonal antibody can be one or more of adalimumab, certolizumab pegol, golimumab, and infliximab.

In various embodiments, the present teachings provide a kit, where the kit includes a therapeutic combination that includes a monoclonal antibody and colchicine. The kit can include one or more containers including the monoclonal antibody and colchicine, typically in separate containers. The kit can include a package insert or label, for example, indicating a treatment and/or instructions for use of the therapeutic combination.

The foregoing as well as other features and advantages of the present teachings will be more fully understood from the following figure, description, example, and claims.

### BRIEF DESCRIPTION OF THE DRAWING

It should be understood that the drawing described below is for illustration purposes only. The drawing is not intended to limit the scope of the present teachings in any way.

FIG. 1 is a graph of a comparison of monoclonal antibody levels in a patient's blood before and after the administration of colchicine.

### DETAILED DESCRIPTION

It now has been discovered that administration of colchicine can increase the time that a monoclonal antibody, such as infliximab, remains in the blood serum of a patient. Unlike known and conventional attempts to increase the effectiveness of monoclonal antibody therapies that can cause the formation of HACAs and/or immunosuppression, the therapeutic combination and combination therapy of the present teachings can mitigate or eliminate one or both of these drawbacks.

Throughout the application, where compositions are described as having, including, or comprising specific components, or where processes are described as having, including, or comprising specific process steps, it is contemplated that compositions of the present teachings also consist essentially of, or consist of the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein,

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of the singular herein, for example, “a,” “an,” or “the,” includes the plural (and vice versa) unless specifically stated otherwise.

Where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

At various places in the present specification, values are disclosed in groups or in ranges. It is specifically intended that the description include each and every individual subcombination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

As used herein, “patient” refers to a mammal, such as a human.

As used herein, a “compound” refers to the compound itself and its pharmaceutically acceptable salts, hydrates and esters, and biologic variations, unless otherwise understood from the context of the description or expressly limited to one particular form of the compound, i.e., the compound itself, or a pharmaceutically acceptable salt, hydrate or ester thereof. A compound of the present teachings can be a monoclonal antibody. Another compound of the present teachings can be colchicine.

Examples of monoclonal antibodies included in the present teachings include, but are not limited to, abciximab, adalimumab, alemtuzumab, atlizumab, basiliximab, belimumab, bevacizumab, brentuximab vecotin, canakinumab, capromab pendetide, catumaxomab, certolizumab pegol, cetuximab, daclizumab, denosumab, eculizumab, edrecolomab, efalizumab, efungumab, ertumaxomab, etanercept, etaracizumab, FBTA05, fontolizumab, gemtuzumab, ozogamicin, girentuximab, golimumab, ibritumomab tiuxetan, igovomab, imciromab, ipilimumab, labetuzumab, mepolizumab, motavizumab, muromonab-cd3, natalizumab, nimotuzumab, nofetumomab merpentan, ofatumumab, omatiztumab, oregovomab, palivizumab, panitumurnab, pemtumomab, pertuzumab, ranibizumab, raxibacumab, rituximab, rovelizumab, ruplizurnab, tacatuzumab tetraxetan, tefibazumab, tocilizumab, tositumomab, trastuzumab, TRBS07, ustekinumab, visilizumab, votumumab, zalutumumab, and zanolimumab. In various embodiments, the monoclonal antibody is infliximab.

As used herein, “therapeutic combination” refers to a combination of one or more active drug substances, i.e., compounds having a therapeutic utility such as a monoclonal antibody and colchicine. Typically, each such compound in the therapeutic combinations of the present teachings can be present in a pharmaceutical composition comprising that compound and a pharmaceutically acceptable carrier. Such compounds can be present in combination amounts that are effective for the intended purpose. The compounds of a therapeutic combination of the present teachings can be administered simultaneously, together or separately, or separately at different times, as part of a regimen.

For example, a combination therapy can be administered as a simultaneous or sequential regimen. When administered sequentially, the combination can be administered in two or more administrations. The combined administration includes co-administration, using separate formulations or a single pharmaceutical formulation, and consecutive administration in either order, where usually there is a time period when both for all) active agents simultaneously exert their biological activities.

Accordingly, in various embodiments, a therapeutic combination can be co-formulated and administered or delivered simultaneously in a combined, unit dosage formulation, in some embodiments, a therapeutic combination can be delivered by alternation or in parallel as separate formulations or by some other regimen. That is, the active compounds can be administered or delivered sequentially, for example, by different injections in separate syringes, separate pills or capsules, or separate infusions.

The present teachings also provide pharmaceutical compositions that include at least one compound described herein such as colchicine or a therapeutic combination, and one or more pharmaceutically acceptable carriers, excipients, or diluents. Examples of such carriers are well known to those skilled in the art and can be prepared in accordance with acceptable pharmaceutical procedures, such as, for example, those described in Remington: The Science and Practice of Pharmacy, 20th edition, ed. Alfonso R. Gennaro, Lippincott Williams & Wilkins, Baltimore, Md. (2000), the entire disclosure of which is incorporated by reference herein for all purposes.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds and therapeutic combinations of the present teachings can be useful for treating a pathological condition or disorder in a patient, for example, a human. As used herein, “treating” refers to partially or completely alleviating and/or ameliorating the condition and/or symptoms thereof. The present teachings accordingly include a method of providing to a patient a pharmaceutical composition that includes a compound or therapeutic combination of the present teachings in combination or association with a pharmaceutically acceptable carrier, Compounds and therapeutic combinations of the present teachings can be administered alone, or in combination with other therapeutically-effective compounds or therapies for the treatment of a pathological condition or disorder. As used herein, “therapeutically-effective” refers to a substance or an amount that elicits a desirable biological activity or effect.

In accordance with the present teachings, the time that a monoclonal antibody remains in the blood serum of a patient can be increased. The method generally includes administering a monoclonal antibody to a patient; and administering colchicine to the patient. In such methods, the monoclonal antibody can remain in the blood serum for a longer period of time in comparison to an absence of colchicine.

in various embodiments, administering colchicine to the patient can be after administering the monoclonal antibody. In particular embodiments administering colchicine to the patient can be contemporaneously with administering the monoclonal antibody. In some embodiments, administering colchicine to the patient can be prior to administering the monoclonal antibody. That is, the monoclonal antibody and colchicine can be administered together or separately, for example, at separate times and in different formulations and/or via different routes of administration. For example, infliximab can be administered intravenously and colchicine can be administered orally. In another example, adalimumab can be administered subcutaneously and colchicine can be administered orally. In some cases, colchicine can be administered intravenously as well.

In certain embodiments, administering a monoclonal antibody can include administering a therapeutically-effective amount of a monoclonal antibody. In particular embodiments, a therapeutically-effective amount of the monoclonal antibody is present in the blood serum for a longer period of time in comparison to an absence of colchicine.

One benefit of the present teachings can be that administering the monoclonal. antibody can occur less frequently in comparison to administering the monoclonal antibody to a patient not being administered colchicine. For example, where a monoclonal antibody is administered such as infused in eight week intervals, the combination therapy of the present teachings can extend the administration of the monoclonal antibody to 10 week or 12 week intervals.

In various embodiments, administering colchicine to the patient can include administering colchicine daily, or every other day, to the patient during monoclonal antibody therapy of the patient. Administering colchicine can begin days or weeks before beginning monoclonal antibody therapy, or can begin contemporaneous with initiating monoclonal antibody therapy, or after such therapy has begun. Colchicine can be administered every third day, every fourth day, or weekly to the patient. In some embodiments, the daily dose of colchicine can be between about 0.05 mg and about 5 mg, between about 0.07 mg and about 3.5 mg, between about 0.08 mg and about 3 mg, or about 0.1 mg and about 2.4 mg.

In various embodiments, administering a monoclonal antibody to the patient can include administering the monoclonal antibody to the patient in intervals of about one week, about two weeks, about three weeks, about four weeks, about five weeks, about six weeks, about seven weeks, about eight weeks, about nine weeks, about 10 weeks, about 11 weeks, or about 12 weeks. That is, the interval between doses of the monoclonal antibody can be about one week to about 12 weeks, including each of the other time intervals disclosed herein.

Suitable dosages for the active compounds such as a monoclonal antibody and/or colchicine can be those dosages presently used, but can be lowered and/or administered less frequently due to the combined action of the active compounds.

In particular embodiments, administering colchicine to the patient can include administering orally colchicine. In certain embodiments, administering the monoclonal antibody to the patient can include administering intravenously the monoclonal antibody, in particular embodiments, administering the monoclonal antibody can include administering subcutaneously the monoclonal antibody. For example, administering infliximab can include administering intravenously infliximab. As another example, administering adalimumab can include administering subcutaneously adalimumab.

In various embodiments of the present teachings, administering colchicine can include administering colchicine in an amount effective to increase the time that the monoclonal antibody remains in the blood serum, for example, comparison to an absence of colchicine. In the present teachings, an absence of colchicine means that the patient is not being administered colchicine and/or any colchicine previously administered to the patient has cleared from the patient's system and is undetectable using current analytical techniques.

In certain embodiments of the present teachings, the methods of increasing the clearance time of a monoclonal antibody from the blood serum of a patient can include disrupting microtubule polymerization or assembly in a patient undergoing monoclonal antibody therapy, whereby a monoclonal antibody concentration in the blood serum of the patient is lengthened compared to not disrupting microtubule polymerization or assembly.

In some embodiments, the present teachings provide methods of increasing the clearance time of a monoclonal antibody from the blood serum of a patient by suppressing nitric oxide synthetase 3 (eNOS3) genes in a patient including monoclonal antibody therapy, whereby a monoclonal antibody concentration in the blood serum of the patient is lengthened compared to not suppressing nitric oxide synthetase 3 (eNOS3) genes.

In particular embodiments, methods of the present teachings can include increasing the clearance time of a monoclonal antibody from the blood serum of a patient. The methods can include decreasing the formation and/or enhancing the removal of human anti-chimeric antibodies in a patient undergoing monoclonal antibody therapy, whereby a monoclonal antibody concentration in the blood serum of the patient is lengthened compared to not decreasing the formation or enhancing the removal of human anti-chimeric antibodies. For example, increasing the clearance time of infliximab from the blood serum of a patient can include decreasing the formation and/or enhancing the removal of HACAs in the patient.

In the methods of the present teachings, the monoclonal antibody concentration can be a therapeutically-effective amount of the monoclonal antibody. However, a therapeutically-effective amount of the monoclonal antibody is not required for colchicine to increase its levels in a patient's bloodstream and/or to reduce the presence of HACAs,

Disrupting microtubule polymerization or assembly, and/or suppressing nitric oxide synthetase 3 eNOS3) genes, and/or decreasing the formation or enhancing the removal of human chimeric antibodies in a patient can include administering a compound to the patient in an amount effective to disrupt microtubule polymerization or assembly, and/or suppress nitric oxide synthetase 3 (eNOS3) genes, and/or decrease the formation or enhancing the removal of human chimeric antibodies in a patient, respectively. The compound can be colchicine.

In any of the methods of the present teachings, the patient can have a monoclonal antibody-treated disorder or condition. The monoclonal antibody-treated disorder or condition can be ulcerative colitis. The monoclonal antibody-treated disorder or condition can be one or more of rheumatoid arthritis, Crohn's disease, plaque psoriasis, psoriatic arthritis, ankylosing spondylitis, juvenile idiopathic arthritis, hemolytic disease of the newborn, inflammatory bowel disease, multiple sclerosis, prevention of organ transplant rejections, non-Hodgkin lymphoma, metastatic cancer, retinopathy of prematurity, ovarian cancer, gastric cancer, head and neck cancer, osteoporosis, paroxysmal nocturnal hemoglobinuria, invasive candida infection, breast cancer, melanoma, chronic lymphocytic leukemia, acute myelogenous leukemia, renal cell carcinoma, colorectal carcinoma, asthma, nasopharyngeal cancer, hemorrhagic shock, staphylococcus aureus infection, and follicular lymphoma.

The monoclonal antibody can be a tumor necrosis factor (TNF) alpha inhibitor. The TNF-alpha inhibitor can be one or more of adalimumab, certolizumab pegol, golimumab, and infliximab.

In particular embodiments, the methods can include increasing the time that a monoclonal antibody remains in the blood serum of a patient such as a human. The method can include administering infliximab to a patient; and administering colchicine to the patient,

In another aspect of the present teachings, a therapeutic combination is provided including a monoclonal antibody such as infliximab and colchicine. The therapeutic combinations can include colchicine and one or more of adalimumab, certolizumab pegol, golimumab, and infliximab.

In various embodiments of the present teachings, an article of manufacture, or “kit”, containing materials useful for the treatment of the diseases and disorders described herein is provided including a therapeutic combination of a monoclonal antibody such as infliximab and colchicine. The kit can include a container including a monoclonal antibody and a container including colchicine. The kit can include a therapeutic combination of colchicine and one or more of adalimumab, certolizumab pegol, golimumab, and infliximab. The kit can include a label or package insert, on or associated with the container(s) or kit. The expression “package insert,” refers to instructions customarily included in commercial packages of therapeutic products that contain information about the indications, usage, dosage, administration, contraindications and/or warnings concerning the use of such therapeutic products.

Suitable containers include, for example, bottles, vials, syringes, blister pack, and the like. The container can be formed from a variety of materials such as glass or plastic. The container can hold a monoclonal antibody and the like or a formulation thereof which is effective, for treating the condition and may have a sterile access port (e.g., the container can be an intravenous solution bag or a vial having a stopper pierceable by a hypodermic injection needle). The label or package insert can indicate that the composition is used for treating the condition of choice, such as an autoimmune disorder. Alternatively, or additionally, the article of manufacture or kit can further include another container including a pharmaceutically acceptable buffer, such as bacteriostatic water for injection (BWFI), phosphate-buffered saline. Ringer's solution and dextrose solution. A kit can further include other materials desirable from a commercial and user standpoint, including other buffers, diluents, filters, needles, and syringes.

A kit can include directions for the administration of a monoclonal antibody and colchicine. For example, the kit can include a first pharmaceutical composition including a monoclonal antibody and a second pharmaceutical composition including colchicine. The kit can include directions for the simultaneous, sequential or separate administration of the first and second pharmaceutical compositions to a patient.

In various embodiments, the kits can be suitable for the delivery of solid oral forms of a compound of the present teachings, for example, colchicine, such as by tablets or capsules. Such a kit can include a number of unit dosages. Such kits can include a card having the dosages oriented in the order of their intended use. An example of such a kit is a “blister pack”. Blister packs are well known in the package industry and are widely used for packaging pharmaceutical unit dosage forms. If desired, a memory aid can be provided, for example, in the form of numbers, letters, or other markings or with a calendar insert, designating the days in the treatment schedule in which the dosages can be administered.

According to same embodiments, a kit can include a first container with a monoclonal antibody contained therein; and a second container with a colchicine contained therein, Alternatively, or additionally, the kit can further include a third container including a pharmaceutically-acceptable buffer, such as bacteriostatic water for injection (BWFI), phosphate-buffered saline, Ringer's solution and dextrose solution. The kit can further include other materials desirable from a commercial and user standpoint, including other buffers, diluents, filters, needles, and syringes.

In certain embodiments, the kit can include a container for containing the separate pharmaceutical compositions such as a divided bottle or a divided foil packet; however, the separate pharmaceutical compositions can be contained within a single, undivided container. Typically, the kit includes directions for the administration of the separate compositions. The kit form can be particularly advantageous when the separate components are administered in different dosage forms (e.g., colchicine orally and a monoclonal antibody parenterally), and/or are administered at different dosage intervals, and/or when titration of the individual components of the therapeutic combination is desired by the prescribing physician.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating, agents, or encapsulating materials. The compounds can be formulated in conventional manner, for example, in a manner similar to that used for known anti-inflammatory agents. Oral formulations containing an active compound disclosed herein can include any conventionally used oral form, including tablets, capsules, buccal forms, troches, lozenges and oral liquids, suspensions or solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkoniurn chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also comprise a compound as described herein in water or fruit juice, containing appropriate solubilizers or emulsifiers as needed.

Liquid carriers can be used in preparing solutions, suspensions, emulsions, syrups, elixirs, and for inhaled delivery. A compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts thereof, in lotions, creams, foams, patches, suspensions, solutions, and suppositories (rectal and vaginal).

When administered for the treatment or inhibition of a particular disease state or disorder, it is understood that an effective dosage can vary depending upon many factors such as the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician or other healthcare provider. The variables involved include the specific condition and its state as well as the size, age and response pattern of the patient.

in some cases, for example those in which the lung is the targeted organ, it may be desirable to administer a compound directly to the airways of the patient, using devices such as metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these active compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, and esters thereof, in lotions, creams, foams, patches, suspensions, solutions, and suppositories (rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

The following example is provided to illustrate further and to facilitate the understanding of the present teachings and is not in any way intended to limit the invention.

### EXAMPLE: PATIENT WITH ULCERATIVE COLITIS

A patient with confirmed ulcerative colitis received monotherapy with the monoclonal antibody infliximab. A therapeutically-effective amount of infliximab was intravenously administered to the patient. The patient developed infusion reactions following infusion of infliximab and was found to have HACAs present based on a serum test. The patient had an infliximab trough level of 2.9 μg/mL in blood drawn before an infliximab infusion which occurred 48 days after the patient's infliximab. After the infusion, the blood serum measured a peak level of 190 μg/mL infliximab.

The patient subsequently developed pain in the joints of the hands, knees and feet, believed to be related to the ulcerative colitis. The patient received an oral administration of 0.6 mg colchicine daily in an effort to treat the joint pain. The patient had an excellent response to colchicine with marked reduction of the joint pain.

The patient's next infusion of infliximab occurred 61 days after the previous infusion. The infliximab trough level measured prior to the infusion was 3.9 which is 34% higher than the previous trough level, despite the blood being drawn and measured 13 days later (i.e., at 61 days vs. 48 days) than the prior infusion.

The presence of HACAs was checked following eight weeks of therapy with colchicine and was found to be negative.

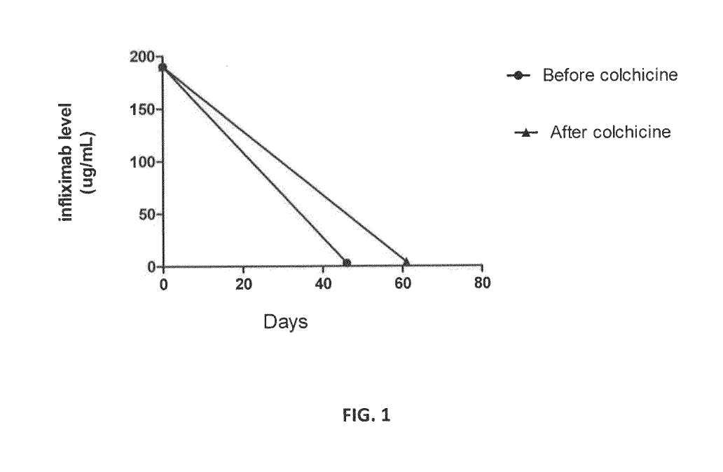

FIG. 1 shows a comparison of infliximab levels in the patient's blood before and after the administration of colchicine. Using a linear model, the estimated time to remove 50% of the infliximab was about 27 days before colchicine treatment and increased by 48% to 40 days after colchicine treatment. Comparing areas under the curve (AUC), these data suggest that colchicine increased the infliximab AUC by 25% (4437 μg*days/mL to 5914 μg*days/mL).

While the above example illustrates one possible method of combination therapy of the present teachings, other variations are possible as described herein.

The present teachings encompass embodiments in other specific forms without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within tie meaning and range of equivalency of the claims are intended to be embraced therein.

What is claimed is:

1. A method of increasing the time that a monoclonal antibody remains in the blood serum of a patient, the method comprising:
administering a monoclonal antibody to a patient; and
administering colchicine to the patient.

2. The method of claim 1, wherein the monoclonal antibody remains in the blood serum for a longer period of time in comparison to an absence of colchicine.

3. The method of claim 1, wherein administering colchicine to the patient is after administering the monoclonal antibody.

4. The method of claim 1, wherein administering colchicine to the patient is prior to administering the monoclonal antibody.

5. The method of claim 1, wherein administering a monoclonal antibody comprises administering a therapeutically-effective amount of a monoclonal antibody.

6. The method of claim 5, wherein a therapeutically-effective amount of the monoclonal antibody is present in the blood serum for a longer period of time in comparison to an absence of colchicine.

7. The method of claim 1, wherein administering the monoclonal antibody can occur less frequently in comparison to administering the monoclonal antibody to a patient not being administered colchicine.

8. The method of claim 1, wherein administering colchicine to the patient comprises administering colchicine daily or every other day to the patient during monoclonal antibody therapy of the patient.

9. The method of claim 8, wherein the daily dose of colchicine is between about 0.1 mg and 2.4 mg.

10. The method of claim 1, wherein administering the monoclonal antibody to the patient comprises at least one of administering intravenously the monoclonal antibody and administering subcutaneously the monoclonal antibody.

11. The method of claim 1, wherein administering colchicine to the patient comprises administering orally colchicine.

12. The method of claim 1, wherein administering colchicine comprises administering colchicine in an amount effective to increase the time that the monoclonal antibody remains in the blood serum.

13. The method of claim 2, wherein an absence of colchicine comprises the patient not being administered colchicine.

14. The method of claim 1, wherein the patient has a monoclonal antibody-treated disorder.

15. The method of claim 14, wherein the monoclonal antibody-treated disorder is selected from one or more of rheumatoid arthritis, ulcerative colitis, Crohn's disease, plaque psoriasis, psoriatic arthritis, ankylosing spondylitis, juvenile idiopathic arthritis, hemolytic disease of the newborn, inflammatory bowel disease, multiple sclerosis, prevention of organ transplant rejections, non-Hodgkin lymphoma, metastatic cancer, retinopathy of prematurity, ovarian cancer, gastric cancer, head and neck cancer, osteoporosis, paroxysmal nocturnal hemoglobinuria, invasive candida infection, breast cancer, melanoma, chronic lymphocytic leukemia, acute myelogenous leukemia, renal cell carcinoma, colorectal carcinoma, asthma, nasopharyngeal cancer, hemorrhagic shock, staphylococcus aureus infection, and follicular lymphoma.

16. The method of claim 1, wherein the monoclonal antibody is a tumor necrosis factor (TNF) alpha inhibitor.

17. The method of claim 16, wherein the TNF-alpha inhibitor is selected from one or more of adalimumab, certolizumab pegol, golimumab, and infliximab.

18. A therapeutic combination comprising a monoclonal antibody and colchicine.

19. A kit comprising:
a therapeutic combination of claim 18; and
a package insert or label.

20. The kit of claim 19, comprising a container comprising the monoclonal antibody and a container comprising colchicine.
